(19) United States
(12) Patent Application Publication
(10) Pub. No.: US 2005/0170383 A1
(43) Pub. Date: Aug. 4, 2005

(54) Microarray hybridization assembly using a heat shrinkage bag for sealing the reaction region

(75) Inventors: Chien-Tsung Wu, Taichung County (TW); Chao-Chi Pan, Hsinchu City (TW); Wen-Yeh Liao, Taiei County (TW); Chih-Wei Ho, Taoyuan County (TW); Yi Yun Chen, Hsinchu City (TW); Shih-Chiao Tseng, Taichung County (TW); Chia Yen Wang, Kaohsiung City (TW); Shyh-Haur Su, Hsinchu County (TW)

Correspondence Address: DERGOSITS & NOAH LLP, FOUR EMBARCADERO CENTER, SUITE 1450, SAN FRANCISCO, CA, 94111, US

(73) Assignee: Phalanx Biotech Group, Inc.

(21) Appl. No.: 10/996,975

(22) Filed: Nov. 23, 2004

Related U.S. Application Data
(63) Continuation-in-part of application No. 10/769,960, filed on Feb. 2, 2004.

(51) Int. Cl. C12Q 1/68; C12M 1/34

(52) U.S. Cl. 435/6; 435/287.2

(57) ABSTRACT

A microarray hybridization assembly for conducting hybridization assay analysis is disclosed. The microarray assembly comprises a slide, a slide cover, and a spacer disposed between the slide and slide cover. The slide cover is placed over the slide and the spacer maintains a gap between the parallel surfaces of the slide and slide cover to create a reaction region for hybridization reactions between attached probe molecules and target molecules of a hybridization solution. The slide assembly is placed into a heat shrinkage bag. The bag and slide assembly are then heated in a heat cycle sufficient to seal the bag around at least three sides of the slide assembly. An optional heat shrinkage cap can be fitted over the fourth side of the slide assembly and heated in a second heat cycle to seal the fourth side of the slide assembly after a hybridization solution is introduced into the reaction region of the slide assembly.

### CROSS REFERENCE TO RELATED APPLICATIONS

The present application is a Continuation-In-Part of U.S. patent application Ser. No. 10/769,960, filed Feb. 2, 2004, entitled “Method of Preparing Reaction Regions for Biochips,” which is currently pending, and which is assigned to the assignee of the present application.

### FIELD OF THE INVENTION

The present invention relates generally to biological and chemical assay systems, and more specifically to microarray hybridization assemblies for use in conducting hybridization assays.

### BACKGROUND OF THE INVENTION

Biological and biochemical analysis often involves the use of glass slides that support hybridization reactions between bound probes and target molecules in a solution. A microarray commonly means a substrate (such as a glass slide, silicon wafer, and nylon or polymer-based substrate) that contains a plurality of different reagents immobilized on the surface. The substrate may have a shape of a rectangle, a square, a circle, a triangle, or any other convenient substantially planar shape. These reagents (known as probes) are usually selected for their high specificity in binding affinity or reactivity toward their counterparts (known as targets) in biological samples. After applying a biological sample onto a microarray under an experimentally-controlled condition, the interactions between each probe on a microarray and its corresponding target in the biological sample can be observed through various target labeling techniques and appropriate detection instrumentation, thus providing the microarray user with qualitative and quantitative information about the target in the tested biological sample.

A key consideration in conducting hybridization assays for a microarray is to provide a reaction region for hybridization reactions between immobilized probe molecules and target molecules in a hybridization solution. Hybridization reactions can often require time periods of up to several to tens of hours and are typically performed at high temperatures for DNA microarrays. Thus, the reaction region must be configured to prevent leaking or drying out of the solution. Several different systems have been developed to accommodate microarray slides for conducting hybridization reactions. For example, U.S. Pat. No. 6,258,593 discloses a slide system in which the slide cover is attached to a substrate through the use of screw fasteners. Such a system is labor intensive and requires the use of special materials and/or production tooling, thus adding a significant complication and expense to the microarray hybridization assay.

It is therefore desirable to provide a simple and economical apparatus and method for assembling slides used in microarray hybridization assay analysis.

### SUMMARY OF THE INVENTION

A microarray hybridization assembly for conducting hybridization assay analysis is disclosed. The microarray assembly comprises a slide, a slide cover, and a spacer disposed between the slide and slide cover. The slide cover is placed over the slide and the spacer maintains a gap between the parallel surfaces of the slide and slide cover to create a reaction region for hybridization reactions between attached probe molecules and target molecules of a hybridization solution. The slide assembly is placed into a heat shrinkage bag. The bag and slide assembly are then heated in a heat cycle sufficient to seal the bag around at least three sides of the slide assembly. A hybridization solution can be introduced through an open end of the slide assembly or an open portion in the gap into the reaction region of the slide assembly. A second heat shrinkage bag, configured as a cap or cover can be fitted over the open end of the assembly and heated in a second heat cycle to seal the fourth side of the slide assembly after a hybridization solution is introduced into the reaction region of the slide assembly.

Other objects, features, and advantages of the present invention will be apparent from the accompanying drawings and from the detailed description that follows below.

### BRIEF DESCRIPTION OF THE DRAWINGS

The present invention is illustrated by way of example and not limitation in the figures of the accompanying drawings, in which like references indicate similar elements, and in which:

FIG. 1A illustrates a heat shrinkage bag suitable for use with embodiments of the present invention;

FIG. 1B illustrates a labeled slide or substrate for use with the heat shrinkage bag shown in FIG. 1A;

FIG. 1C illustrates a spacer for separating a slide and slide cover, according to one embodiment of the present invention;

FIG. 1D illustrates a slide cover for use with the labeled slide shown in FIG. 1B;

FIG. 2A illustrates a top view of a slide and slide cover assembly placed within a heat shrinkage bag, according to one embodiment of the present invention; and

FIG. 2B illustrates a cross-sectional view of the slide and slide cover assembly of FIG. 2A;

FIG. 3 illustrates a method of heating a heat shrinkage bag around a slide assembly, according to one embodiment of the present invention;

FIG. 4A illustrates a top view of a slide assembly sealed within a heat shrinkage bag, according to one embodiment of the present invention;

FIG. 4B illustrates a cross-sectional view of the bag and slide assembly of FIG. 4A;

FIG. 5 illustrates the introduction of a hybridization solution into a reaction region of a slide assembly, according to one embodiment of the present invention;

FIG. 6A illustrates a spacer for separating a slide and slide cover, according to a first alternative embodiment of the present invention; and

FIG. 6B illustrates a spacer for separating a slide and slide cover, according to a second alternative embodiment of the present invention.

### DETAILED DESCRIPTION OF THE PREFERRED EMBODIMENT

The present invention discloses a microarray hybridization assembly that uses a heat shrinkage bag to fit and seal the hybridization assembly for use in hybridization reaction processes. The microarrays include, but are not limited to, gene chips, DNA chips, oligonucleotide microarrays, polynucleotide microarrays, protein microarrays, antibody microarrays. For purposes of the following description the terms “hybridization assembly” or “slide assembly” refer to a structure comprising a slide cover mounted on a slide substrate with a spacer placed between them to form a reaction region that can hold a hybridization solution as a heat shrinkage bag is fitted around the slide assembly and then shrunk to form a hermetically sealed reaction region. In the following description, for purposes of explanation, numerous specific details are set forth in order to provide a thorough understanding of the present invention. It will be evident, however, to one of ordinary skill in the art, that the present invention may be practiced without these specific details. In other instances, well-known structures and devices are shown in block diagram form to facilitate explanation. The description of preferred embodiments is not intended to limit the scope of the claims appended hereto.

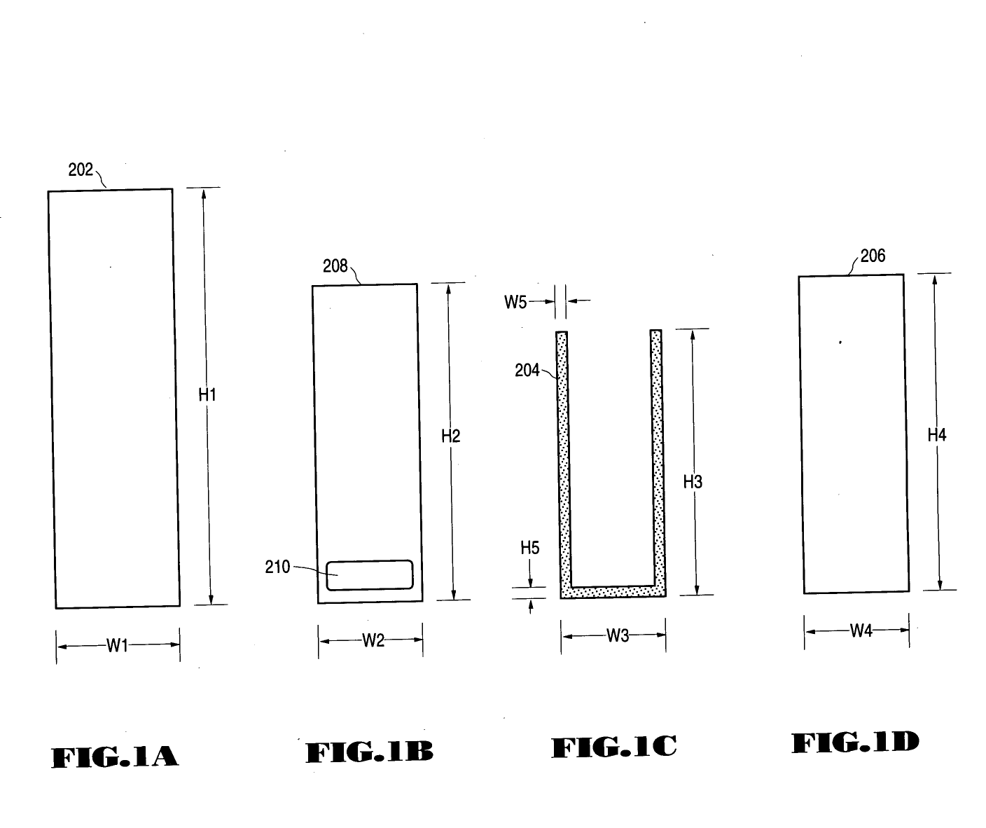

FIGS. 1A through 1D illustrate the component parts of a hybridization assembly according to one embodiment of the present invention. The illustrated hybridization assembly is suitable for use in encasing a reaction region for a hybridization solution for target molecules, such as nucleic acid, polynucleotide, oligonucleotide, mRNA, cDNA, or the like. FIG. 1B is a slide or similar substrate 208 for attachment of an array of probe molecules (e.g., oligonucleotides). The substrate 208 can be made of glass, plastic, fused silica, silicon, ceramic, metal or similar material. The dimensions of slide 208 may be varied to accommodate different uses, but for typical oligonucleotide microarray applications, the dimensions of slide 208 are typically on the order of width (W2):25 mm, and height (H2): 75 mm. This represents a slide of approximate dimensions of 1×3 inches. Slide 208 may also include a label 210 that allows the marking or identification of the sample carried by the slide. FIG. 1D illustrates a slide cover or similar substrate 206 for use with slide 208. The slide cover 206 acts to provide an upper surface for the reaction area defined by the slide and the slide cover, and may be made of the same or similar material as the slide itself, e.g., glass, plastic, and so on. In a preferred embodiment of the present invention, the slide cover 206 has dimensions W4×H4 that are identical to that of the slide 208 so that both the slide and slide cover are the same size. In an alternative embodiment, the slide cover 206 may be made smaller than the slide by reducing the width and/or height relative to the slide. In addition, the slide cover 206 itself may have or may not have probe molecules immobilized on its surface. That is, it can be a microarray, or a substrate without probe molecules immobilized on its surface.

A reaction region enclosing the attached probes on the surface of slide 208 is created by mounting the slide cover 206 onto the slide 208 and separating the two mating surfaces by a small gap so that two parallel upper and lower surfaces are created. FIG. 1C illustrates a spacer 204 that is disposed between the slide 208 and slide cover 206, according to one embodiment of the present invention. The spacer can be a solid or flexible material, which is thermally stable and chemically inert. The spacer should remain substantially unchanged by contact with a hybridization solution, and substantially unchanged at temperatures in excess of 50° C., and preferably up to temperatures of about 90° C. The material comprising the spacer 204 can be rubber, silicon rubber, plastic, gel, metal, or similar material. For the embodiment illustrated in FIG. 1C, the spacer 204 is substantially flat and three sided, and has a thickness of about 80 to about 400 micron.

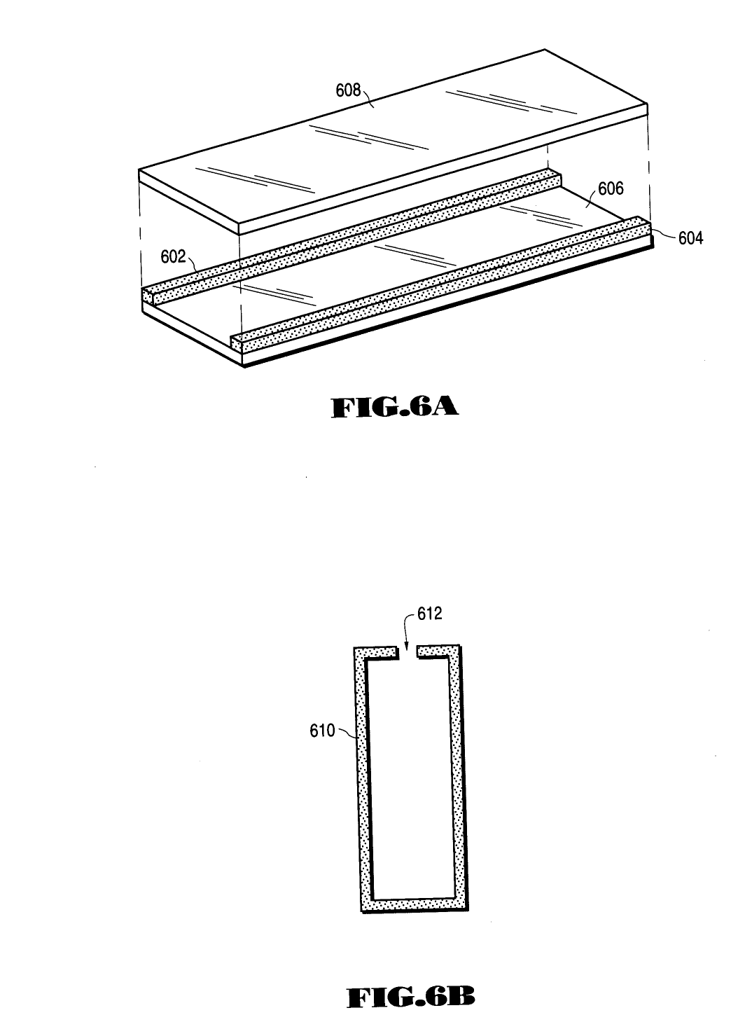

It should be noted that the spacer 204 can be of various different forms from that shown in FIG. 1C. For example the spacer can comprise a plurality (e.g., two to four) of separate unattached sections that are configured to be placed around the borders of the slide. FIG. 6A illustrates a spacer for separating a slide and slide cover, according to a first alternative embodiment of the present invention, in which the spacer comprises two separate sections 602 and 604, which are placed between a substrate 606 and a slide or second substrate 608. FIG. 6B illustrates a spacer for separating a slide and slide cover, according to another alternative embodiment of the present invention. In this alternative embodiment, the spacer 610 is a four sided spacer in which a portion of one side has an opening that allows the introduction of a target solution into the reaction region defined by the walls of the spacer.

Generally the spacer is placed around the outer edge of the slide 208 of the microarray, the spacer can be affixed around the outer edge of the slide 208, to create a reaction region for hybridization on the substrate surface when the slide cover 206 is mounted on the slide 208. As illustrated in FIG. 1C, the dimensions W3×H3 of the spacer 204 are typically defined so that the spacer fits just within the outside dimensions of the slide 208 and slide cover 206. Thus, for the embodiment illustrated in FIG. 1C, the overall height of the spacer, H3, can be less than or equal to the height of the slide H2, and the overall width of the spacer, W3, can be equal to or smaller than the width of the slide W2. If the slide cover 206 is smaller than the slide 208, the dimensions of the spacer should be tailored to match the dimensions of the slide cover. In an alternative embodiment, the spacer is placed and affixed around the outer edge of the slide cover 206 to create a reaction region when slide cover 206 is mounted on the slide 208. The spacer can be applied to the slide cover by an adhesive material before it is placed on the slide cover. In such an embodiment, the composition of the spacer 204 can be silicon rubber, on which one surface has been applied silicone sealant. The spacer can then be stuck onto and around the outer edge of the slide cover 206.

The region of the spacer contained within its three walls defines the reaction area of the hybridization assembly. The spacer height, H5, is typically on the order of 3 to 10 mm, and the spacer width, W5, is typically on the order of 3 to 5 mm. In general, the spacer may have any suitable shape or dimension as long as the spacer does not cover any portion of the probe area of the microarray and a suitable volume of the reaction region can be created. In addition, the spacer may be configured to be placed proximately around an inside portion of at least two outer edges of the slide and the slide cover. For most typical oligonucleotide hybridization reactions, the volume of the reaction region is on the order of about 100 μl to about 500 μl. This volume should be carefully calibrated, depending upon the application. If the reaction volume is too small, due to too thin a spacer, it may complicate the hybridization treatment. If the reaction region volume is too big, the concentration of target sample molecule is decreased, and so therefore is the sensitivity of hybridization assays.

FIG. 1A illustrates a heat shrinkage bag that is used to enclose a slide, spacer, and slide cover assembly, according to one embodiment of the present invention. The heat shrinkage bag 202 is configured to fit around the slide and slide cover assembly and shrink around the assembly when the bag is heated under proper conditions, thus sealing the assembly within a hermetically sealed environment. Thus, the bag 202 is made of a material that features shrinkage in two dimensions upon the proper application of heat. In one embodiment, the bag 202 is formed by a heat shrinkage film made of polyvinylchloride (PVC), polypropylene (PP), polyethylene (PE), polyethyleneterephthalate (PET), polytetrafluoroethylene (PTFE), polyolefine (POF), or similar type of material. Other heat shrinkage materials can also be used to form the bag 202, such as heat shrinkage films of Neoprene, Teflon, Nylon, Urethane, Silicone, and so on. For applications in which the slide assembly is deconstructed after incubation and prior to analysis, the bag can be made of a transparent, translucent, or opaque material. For applications in which analysis is performed while the slide assembly is sealed within the bag, the bag should be made of a transparent or substantially transparent material.

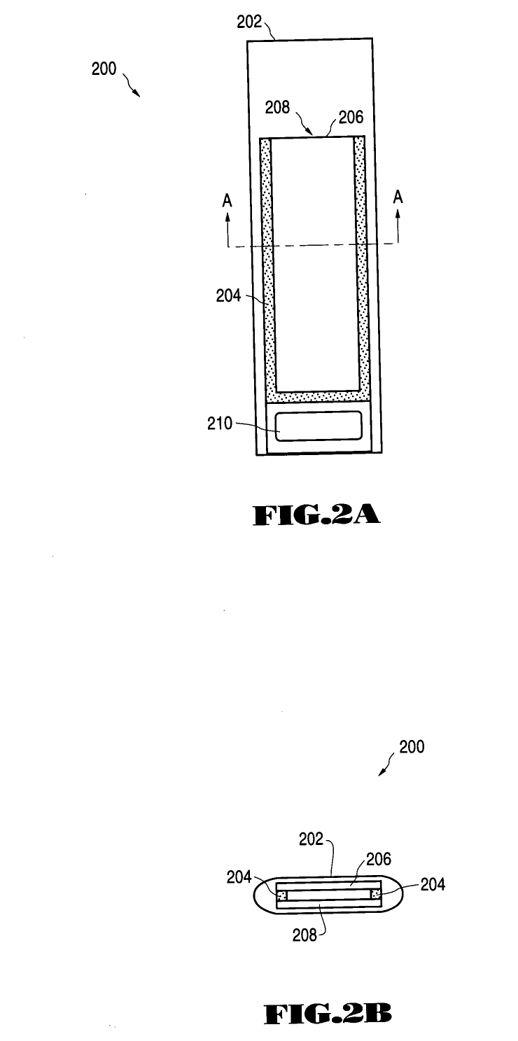

FIG. 2A illustrates the slide, spacer, and slide cover assembly of FIGS. 1B, 1C, and 1D placed within the heat shrinkage bag 202, according to one embodiment of the present invention. FIG. 2B shows a cross-sectional view of the slide assembly within the bag prior to shrinkage of the bag.

As illustrated in FIGS. 2A and 2B, the bag 202 can be large enough to comfortably fit around the slide assembly, yet tight enough to ensure a sealed fit when the bag is shrunk around the assembly. Once the slide assembly is placed in the heat shrinkage bag, it can be lowered to the bottom of the bag and positioned approximately in the middle of the bag in the horizontal direction, to have a tight seal around the slide assembly. The enclosed slide assembly is then subject to a heating cycle to shrink the bag. The temperature and time duration required for the heat cycle depends upon the material used for the bag. In general, the bag can be heated to a temperature sufficient to shrink the bag around the slide assembly by placing the bag in hot water, baking in an oven, directing a hot gas over the bag, or any similar heating method.

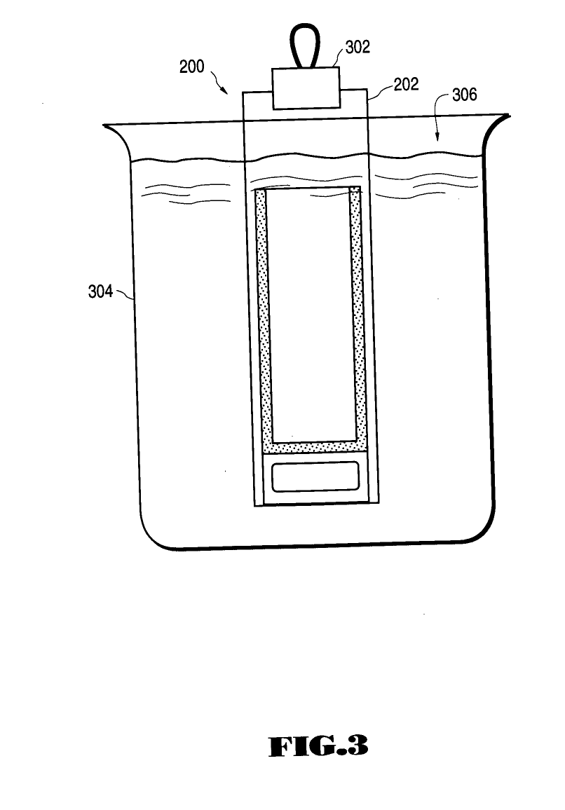

FIG. 3 illustrates the step of heating the heat shrinkage bag around the slide assembly for an embodiment in which the bag 202 is a polyvinylchloride heat shrinkage film bag. For this embodiment, the bag and slide assembly 200 is sealed with a binder clip or similar fastening means 302 and placed in a container 304 of hot water 306. The temperature of the water can be on the order of 90° C. for a period of time sufficient to shrink the bag around the slide assembly, e.g., 0.5 to 2 minutes, depending upon the type and thickness of the bag material.

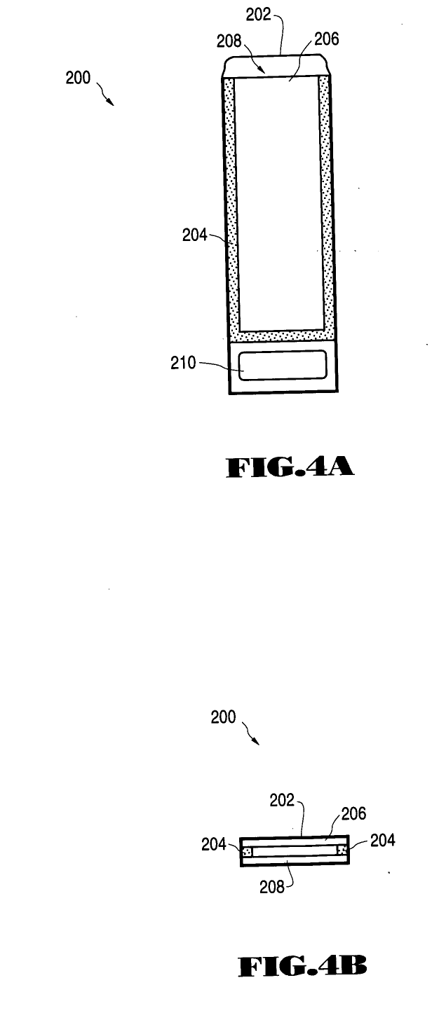

Upon shrinking, the slide assembly can be sealed within the bag as shown in FIGS. 4A and 4B. FIG. 4A shows the bag 202 tightened and sealed around at least three sides of the slide 208 and slide cover 206 assembly. FIG. 4B shows a cross-section of the slide assembly of FIG. 4A and illustrates the gap created by spacer 204 between the slide 208 and slide cover 206.

In one embodiment, the heat shrinkage bag 202 can be sized so that sufficient material is available at the top (open end) of the bag when the bag is immersed in water, such that adequate surface area is available to grip the bag using fastener 302 and such that water does not enter the bag during the sealing process. The bag should be placed sufficiently low in the water so that the whole height H2 of the two sides of slide 208 is sealed during the heating cycle. The excess top portion of the bag above the top (open end) of the slide assembly can then be cut away.

The slide assembly described with reference to FIGS. 1A through 1D can be used to encase a sample or solution that is introduced into the reaction region of the assembly after sealing of the assembly by the bag. For this embodiment, the assembly is constructed by first positioning the slide with the target (e.g., microarray probe) side up, aligning the spacer on the microarray, placing the blank slide cover on the top of the spacer and aligning the slide and slide cover to overlap each other. The reaction region is then created between the microarray probe surface and the blank slide surface. This hybridization assembly is placed into the heat shrinkage bag with a side of the bag left open to accommodate the introduction of the assembly into the bag. The assembly is then heated to shrink and wrap three sides of the slide and slide cover tightly together. Any excess bag material from the open (sample loading) side of the assembly can be trimmed off. The target hybridization solution can then be added to the reaction region of the hybridization assembly through open sample loading side of the assembly. In most cases it is preferable to fill the reaction region at least to cover a probe region. In addition, the reaction region can be filled to almost full capacity. For one embodiment, the spacer that is placed between the slide and slide cover can have three sides, as illustrated in FIG. 1C. If a spacer with more than three sides is used, at least one of the sides should contain an opening 612 to allow introduction of a solution into the reaction region. Such a spacer is illustrated in FIG. 6B.

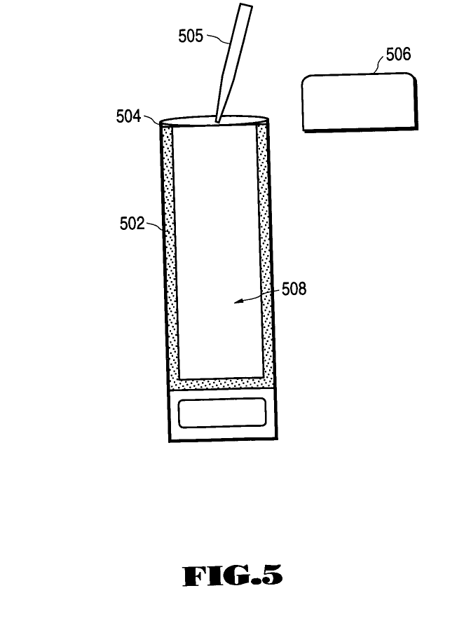

FIG. 5 illustrates an embodiment in which a target hybridization solution is added to the reaction region of the hybridization assembly through an open sample loading side of the assembly. After the heating cycle, three sides of the slide assembly around the spacer walls 502 are sealed. The excess portion of the bag along the top side of the assembly can be then cut. This creates an open slot 504 coincident to the open portion of the spacer 502. A target solution can then be introduced into the reaction region 508 of the slide assembly through the open portion of the spacer 502 by way of a pipette 505 or similar instrument.

For the embodiment illustrated in FIG. 5, a second heat shrinkage bag in the form of a heat shrinkage “cap” can be used to seal the top (open end) of the slide assembly once the bag has sealed around the slide assembly and a hybridization solution has been introduced into a reaction region of the slide assembly. As shown in FIG. 5, heat shrinkage cap 506 is used to seal the top portion (open end) 504 of the slide assembly. The heat shrinkage cap 506 can be of any size sufficient to fully seal the open end of the slide assembly. For example, it can be smaller (shorter) than the original heat shrinkage bag, as shown in FIG. 5, or it can be the same size or larger than the original bag. Furthermore, the heat shrinkage cap can be a separately formed item from the original bag, or it can be a portion of the original bag that is cut away from the bag. This heat shrinkage cap is sealed around the slide assembly in a second heat cycle after the original bag seals the slide assembly. The heat shrinkage cap can be shaped so that it is tightly friction fitted around the open end of the slide assembly prior to the second heat cycle, or it can be held in place by adhesive means. Any excess portion of the heat shrinkage bag can be cut away after the second heat cycle if there is any excess portion of the bag.

The heat shrinkage cap embodiment is typically used in applications in which the hybridization solution is introduced into the slide assembly after the first heat shrinkage obag is sealed around the slide assembly to form the reaction chamber. This application is shown in FIG. 5 wherein the top portion of bag 504 is open at least along a portion of the width of the slide assembly. A hybridization solution is pipetted or otherwise introduced into the slide assembly through the open end of the slide assembly. In this manner, the hybridization solution is introduced into the reaction chamber 508 of the slide assembly.

Once the slide assembly is filled with the target hybridization solution and sealed with the first heat shrinkage bag and the heat shrinkage cap, it can be incubated under hybridization conditions in accordance with the requirements of the interaction of probe molecules and target molecules. After incubation, the array can be washed and read through suitable means.

In typical applications, the assembly may be disassembled after the hybridization reaction is complete, so that the slide with the hybridized target molecules on its surface is analyzed directly. For these applications, the bag material is removed from the hybridization assembly after incubation.

As described with reference to the embodiment illustrated in FIGS. 1A through 1D, the slide cover 206 is a blank cover that fits over the slide substrate 208. In an alternative embodiment, the slide cover can be a second micro array instead of a blank slide. This second microarray can be placed on the top of the first microarray on the slide, such that the probe containing surface of the second microarray faces the probe containing surface of the first microarray. The second microarray can be the same as or different from the first microarray. The dimensions provided above with respect to the slide, slide cover, spacer, heat shrinkage bag, and heat shrinkage cap are meant to be illustrative in nature and not limiting as to the scope of the claimed invention. The components illustrated in FIGS. 1A through 1D can be provided in many different dimensions, and particular dimensions can be varied to meet the constraints and requirements of the assay systems being utilized. Furthermore, the size and orientation of the probe surface and reaction regions on the surface of the slide depend upon the particular application and can vary depending upon specific embodiments.

### EXAMPLE 1

The following example illustrates the steps taken to prepare a hybridization assembly and conduct a hybridization assay with the method and techniques described above.

(A) Preparation of Hybridization Solution:

HepG2 (ATCC No. HB-8065)cell was grown on a laboratory dish in ATCC (American Type Culture Collection) complete growth medium at 37° C. to a cell number of 6×10⁶. The cell was then harvested for total RNA extraction. The total RNA was extracted using Qiagen RNeasy Midi Kit (Catalogue No. 75144). After extraction, 20 kg of the total RNA was taken and converted to Cy5 labeled cDNA according to Agilent Fluorescent Direct label Kit (Catalogue No.G2557A) in each run of a total of ten runs. In each run, a Perkin-Elmer Cy5-dCTP reagent was used for direct labeling, and a Qiagen PCR Purification Kit (Catalogue No. 28106) was applied to provide purified fluorescent labeled cDNA elution. The elution was concentrated using Millipore YM30 filter (Catalogue No. 42410) so that one μl of the final cDNA solution corresponded to 2.5 μg of total RNA converted in the run. In this manner, 200 μg of total RNA was converted to 80 μl of fluorescent labeled cDNA solution. The following steps were taken to prepare a target hybridization mix, target sample solution:

(1) A water bath was preheated to about 90° C.

(2) The 1.5× hybridization buffer (7.5×SSC, 45%Foramide, 0.15%BSA, 1.5 mM EDTA, 0.75%SDS) was agitated in a 65° C. water bath for 10 minutes.

(3) 25 μl of the fluorescent labeled cDNA and 173 μl of the 1.5× hybridization buffer were mixed, and nuclease-free ddH₂O was added to bring up a total volume of 260 μl of target hybridization mix comprising the fluorescent labeled cDNA hybridization mix.

(4) A denature program was set in a PCR machine to 95° C. for 5 minutes, then to a steady temperature of 60° C. The target hybridization mix was placed in the PCR machine and the Denature program was started. While the hybridization mix was denaturing, a hybridization assembly was prepared according to step B.

(B) Preparation of Hybridization Assembly:

(1) A glass microarray slide and glass slide cover each having dimensions on the order of of 2.5 cm×7.6 cm (approximately 1 inch×3 inch) were obtained. The slide comprised an oligonucleotide microarray slide containing a plurality of oligonucleotides attached to the surface of the slide. A spacer made of silicon rubber was cut to a three sided shape for placement between the slide and slide cover as shown in FIG. 1C. The spacer had a thickness of 0.21 mm, a width W3 of 25 mm, a height H3 of 64 mm, a segment width W5 of 2.5 mm, and a segment height H5 of 2.5 mm.

(2) The microarray probe side of the slide was positioned upwards and the spacer was placed on the upper surface of the slide with its corners aligned with two corners of the microarray. The blank slide cover was placed on the top of the spacer and the edges of the two slides were aligned to form a hybridization assembly, such as that shown in FIG. 2A. A reaction region was created between the microarray probe surface and the blank slide surface.

(3) The hybridization assembly was fitted into a polyvinylchloride heat shrinkage bag, and the thickness of the bag material was on the order of 0.04 mm. The bag was cut to a size of about 30 mm×100 mm. The sample loading side of the hybridization assembly faced the opening side of the bag. The assembly was lowered to the end of the bag, then the bag opening side was clipped with a binder clip.

(4) The clipped assembly of the slide assembly in the heat shrinkage bag was immersed into a beaker of water heated to about 90° C. for about 10-20 seconds to shrink and wrap the slides inside the bag. After shrinkage, the bag was lifted out of the water, and the clips were removed. The portion of the excess bag was trimmed off the sample loading side of the assembly.

(C) Addition of Target Sample Solution to the Hybridization Assembly:

(1) After production of the hybridization assembly with the open bag side, the assembly was preheated in a 50° C. oven for more than 10 minutes. About 260 μl of the fluorescent labeled cDNA hybridization mix prepared in step (A) was added to the reaction region through the open side of the hybridization assembly.

(2) The sample loading side of the hybridization assembly was covered with a polyvinylchloride heat shrinkage cap, which was cut to a size of about 30 mm×7 mm. The thickness of the cap material was on the order of 0.04 mm. The cap was held in place through a friction fit, and the capped side of the assembly was immersed in 90° C. hot water to seal the sample loading opening of the assembly. After sealing of the assembly end, the entire assembly was immersed in the hot water bath to completely seal the assembly in the polyvinylchloride heat shrinkage film.

(D) Hybridization Reaction and Analysis:

(1) After sealing, the hybridization assembly was placed in a 50° C. oven with rotation (3 rpm) for 14-16 hours for incubation of the hybridization reaction. The oven was kept humidified with 2×SSC.

(2) After incubation, an incision was made along the edge of the glass slides with a craft knife to open the wrapped hybridization assembly. The wrapped film was removed and the hybridization assembly was entirely submerged in 42° C. 2×SSC, 0.2% SDS solution. The assembly was disassembled in the solution.

(3) The microarray was washed with excess amount of pre-warmed 2×SSC, 0.2% SDS for ten minutes at 42° C.

(4) The microarray was then washed with excess amount of pre-warmed 2×SSC for ten minutes at 42° C.

(5) The microarray was finally washed with excess amount of 0.2×SSC for ten minutes at room temperature, and then dried in a spin dry centrifuge.

(6) The microarray was scanned by Axon Genepix 4000B scanner and analyzed by fluorescence detection to quantify the amount of cDNA hybridized with each oligonucleotide probe of the microarray.

Further, a polyethyleneterephthalate heat shrinkage bag with dimensions on the order of 30 mm×100 mm and a thickness of the bag material on the order of 0.06 mm worked similarly in the first heating cycle as the polyvinylchloride heat shrinkage bag of the Example 1. A polyethyleneterephthalate heat shrinkage cap with dimensions on the order of 30 mm×7 mm and a thickness of the bag material on the order of 0.06 mm also worked similarly in the second heating cycle as the polyvinylchloride heat shrinkage cap of the Example 1.

In the foregoing, a slide system for microarrays has been described. Although the present invention has been described with reference to specific exemplary embodiments, it will be evident that various modifications and changes may be made to these embodiments without departing from the broader spirit and scope of the invention as set forth in the claims. For example, although embodiments of the invention were described with reference to use with oligonucleotide or polynucleotide arrays, it should be noted that the use of a heat shrinkage bag and a spacer to produce a sealed reaction region between two laboratory slides can be used for analysis of many other different biological or chemical substances, such as protein arrays, antibody arrays, and the like. Accordingly, the specification and drawings are to be regarded in an illustrative rather than a restrictive sense.

1. An apparatus for conducting hybridization reactions, comprising:
a first substrate comprising a surface;
a substantially parallel second substrate having a surface facing the surface of the first substrate;
a spacer aligned between the surface of the first substrate and the surface of the second substrate, wherein the spacer is placed proximately around an inside portion of at least two outer edges of the first substrate surface and the second substrate surface, the spacer creating a reaction region and an open portion between the first substrate and the second substrate, wherein the open portion allows introduction of a solution into the reaction region; and
a heat shrinkage bag shrunk to hermetically seal the first substrate and the second substrate around at least two sides of the first and second substrates, the bag configured to enclose the first substrate, second substrate, and spacer when subject to ambient temperatures before shrinkage of the bag.

2. The apparatus of claim 1, wherein the surface of the first substrate contains a plurality of oligonucleotide probes.

3. The apparatus of claim 1, wherein the first substrate and the second substrate are individually comprised of a material selected from the group consisting of glass, fused silica, silicon, plastic, ceramic, and metal.

4. The apparatus of claim 1, wherein the spacer comprises a three sided structure including three connected segments.

5. The apparatus of claim 1, wherein the spacer comprises a material of a thickness of about 80 to about 400 microns.

6. The apparatus of claim 1, wherein the spacer is comprised of a material selected from the group consisting of rubber, metal, gel, and plastic.

7. The apparatus of claim 1, wherein the heat shrinkage bag is comprised of a material selected from the group consisting of heat shrinkage films of neoprene, teflon, nylon, urethane, silicone, polyvinylchoride, polypropylene, polyethylene, polyethyleneterephthalate, polytetrafluoroethylene, and polyolefine.

8. The apparatus of claim 1, wherein the bag has an open slot allowing introduction of a hybridization solution into the reaction region.

9. The apparatus of claim 1, further comprising a heat shrinkage cap shrunk to hermetically seal an open end of an assembly of the first substrate, second substrate, and spacer, the cap configured to cover the open end of the assembly at ambient temperatures before shrinkage of the cap.

10. The apparatus of claim 9, wherein the heat shrinkage cap is comprised of a material selected from the group consisting heat shrinkage films of neoprene, teflon, nylon, urethane, silicone, polyvinylchoride, polypropylene, polyethylene, polyethyleneterephthalate, polytetrafluoroethylene, and polyolefine.

11. The apparatus of claim 1, wherein the surface of the second substrate contains a plurality of oligonucleotide probes.

12. The apparatus of claim 1, further comprising a hybridization solution comprising a target molecule which hybridizes to a surface-attached probe molecule within the reaction region.

13. The apparatus of claim 1, wherein the surface of the second substrate contains a plurality of polynucleotide probes.

14. The apparatus of claim 1, wherein the surface of the first substrate contains a plurality of polynucleotide probes.

15. The apparatus of claim 4, wherein the spacer comprises a thickness of about 0.2 to about 0.3 mm, a width W3 of about 25 mm of one side, a height H3 of about 64 to about 76 mm of two sides, a segment width W5 of about 2.5 to about 4 mm, a segment height H5 of about 2.5 to about 5 mm, and the first substrate and the second substrate have substantially same rectangular dimensions.

16. The apparatus of claim 15, wherein the thickness is about 0.21 mm, the segment width W5 is about 2.5 mm, the segment height H5 is about 2.5 mm.

17. The apparatus of claim 4, wherein the spacer is comprised of a material selected the group consisting of rubber, metal, gel, and plastic.

18. The apparatus of claim 15, wherein the spacer is comprised of a material selected the group consisting of rubber, metal, gel, and plastic.

19. The apparatus of claim 16, wherein the spacer is comprised of a material selected the group consisting of rubber, metal, gel, and plastic.

20. The apparatus of claim 7, further comprising a heat shrinkage cap shrunk to hermetically seal an open end of an assembly of the first substrate, second substrate, and spacer, the cap configured to cover the open end of the assembly at ambient temperatures before shrinkage of the cap.

21. The apparatus of claim 20, wherein the heat shrinkage cap is comprised of a material selected from the group consisting of heat shrinkage films of neoprene, Teflon, nylon, urethane, silicone, polyvinylchoride, polypropylene, polyethylene, polyethyleneterephthalate, polytetrafluoroethylene, and polyolefine.

22. The apparatus of claim 1, wherein the spacer is affixed to the surface of the first substrate or the second substrate.

23. The apparatus of claim 2, wherein the spacer is affixed to the surface of the second substrate.

24. The apparatus of claim 14, wherein the spacer is affixed to the surface of the second substrate.

25. The apparatus of claim 11, wherein the spacer is affixed to the surface of the first substrate.

26. The apparatus of claim 14, wherein the spacer is affixed to the surface of the first substrate.

27. The apparatus of claim 1, wherein the first substrate and the second substrate have substantially rectangular dimensions, and at least three sides of the first substrate and the second substrate are hermetically sealed.

28. A heat shrinkage bag for wrapping around a hybridization assembly, comprising a material selected from the group consisting of heat shrinkage films of neoprene, teflon, nylon, urethane, silicone, polyvinylchloride, polypropylene, polyethylene, polyethyleneterephthalate, polytetrafluoroethylene, and polyolefine; the bag having a substantially rectangular shape and an open side, and configured to enclose an assembly of a first substrate, second substrate, and spacer at an end of the bag at ambient temperatures, and shrink to hermetically seal at least two sides of the assembly when subject to an elevated temperature, the first and second substrates having substantially rectangular dimensions, and including a plurality of oligonucleotide or polynucleotide probes on at least one surface of the first or second substrate.

29. The heat shrinkage bag of claim 28, wherein the material is a heat shrinkage film of polyvinylchloride (PVC) or polyethyleneterephthalate (PET), the heat shrinkage bag has one dimension of about 30 mm and another dimension of about 82 to about 100 mm or about 8 to about 80 mm, and the elevated temperature step comprises immersing the assembly into hot water of about 90° C.

30. A spacer for creating a reaction region between a first substrate and a second substrate, comprising a three sided structure including three connected segments; the spacer having a substantially uniform thickness and configured to be aligned between a surface of the first substrate and a surface of the second substrate, and placed proximately around an inside portion of at least two outer edges of the first substrate surface and the second substrate surface to create a reaction region and an open portion allowing introduction of a solution into the reaction region there between, the first and second substrates having substantially rectangular dimensions, and including a plurality of oligonucleotide or polynucleotide probes on at least one surface of the first or second substrate.

31. The spacer of claim 30, further comprising a material selected from the group consisting of rubber, metal, gel, and plastic.

32. The spacer of claim 30, wherein the three sided structure comprises a thickness of about 0.2 to about 0.3 mm, a width W3 of about 25 mm of one side, a height H3 of about 64 to about 76 mm of two sides, a segment width W5 of about 2.5 to about 4 mm, and a segment height H5 of about 2.5 to about 5 mm.

33. A method comprising the steps of:
providing a first substrate comprising a surface;
placing a spacer having a substantially uniform thickness proximate an inside portion of at least two outer edges of the surface of the first substrate;
mounting a surface of a second substrate onto the spacer such that the surface of the first substrate is substantially parallel to the surface of the second substrate, thereby creating a reaction region and an open portion there between, wherein the open portion allows introduction of a solution into the reaction region;
fitting an assembly of the first substrate, spacer, and second substrate into a heat shrinkage bag; and providing heat to shrink the bag to form a substantially hermetic seal around a side of the assembly.

34. The method of claim 33, wherein the heat shrinkage bag has sufficient excess materials above an open edge of the assembly, and the heating step comprises immersing the heat shrinkage bag into water heated to a temperature exceeding 50° C.

35. The method of claim 33, wherein the heat shrinkage bag is comprised of polyvinylchloride (PVC) heat shrinkage film or polyethyleneterephthalate (PET) heat shrinkage film.

36. The method of claim 33, wherein the heat shrinkage bag is comprised of a material selected from the group consisting of heat shrinkage films of neoprene, teflon, nylon, urethane, silicone, polypropylene, polyethylene, polytetrafluoroethylene and polyolefine.

37. The method of claim 33, wherein the first substrate includes a plurality of molecules selected from the group consisting of oligonucleotides and polynucleotides.

38. The method of claim 34 further comprising the steps of:
removing the excess material along the open edge of the assembly; and
introducing a hybridization solution comprising a target molecule which hybridizes to a surface-attached probe molecule into the reaction region.

39. The method of claim 38 further comprising the steps of:
sealing the open edge of the assembly; and
incubating the hybridization solution under hybridization conditions.

40. The method of claim 39 wherein the step of sealing the open edge comprises the substeps of:
fitting a heat shrinkage cap over the open edge of the assembly; and
immersing the open end of the assembly containing the heat shrinkage cap into water heated to a temperature exceeding 50° C.

41. The method of claim 33, wherein the spacer is affixed to an inside portion of the surface of the first substrate or the second substrate.

42. A kit for conducting hybridization assay analysis, comprising:
a first substrate;
a second substrate for placement proximately over the first substrate;
a spacer for placement between the first substrate and second substrate, the spacer configured to be placed proximately around an inside portion of at least two outer edges of the first substrate and the second substrate, the spacer creating a reaction region and an open portion between the first and second substrates, wherein the open portion allows introduction of a solution into the reaction region; and
a heat shrinkage bag configured to enclose the first substrate, second substrate, and spacer in one orientation when subject to ambient temperatures, and to tightly seal the first and second substrates around a side of the first substrate and second substrate when subject to an elevated temperature.

43. The kit of claim 42 further comprising a heat shrinkage cap configured to enclose the first substrate, second substrate and spacer in another orientation when subject to ambient temperatures, and to tightly seal an open end of an assembly of the first substrate, second substrate, and spacer when subject to an elevated temperature.

44. The kit of claim 42, wherein the first substrate and second substrate are each comprised of a material selected from the group consisting of glass, fused silica, silicon, plastic, ceramic, and metal.

45. The kit of claim 44, wherein at least a surface of the first substrate or the second substrate contains a plurality of oligonucleotide or polynucleotide probes.

46. The kit of claim 42, wherein the spacer is comprised of a material selected from the group consisting of rubber, metal, gel, and plastic.

47. The kit of claim 46, wherein the spacer comprises a three sided structure including three connected segments.

48. The kit of claim 46, wherein the spacer comprises two separate segments configured to be placed along opposite side edges of the first substrate.

49. The kit of claim 42, wherein the heat shrinkage bag is comprised of a material selected from a group consisting of heat shrinkage films of neoprene, teflon, nylon, urethane, silicone, polyvinylchloride, polypropylene, polyethylene, polyethyleneterephthalate, polytetrafluoroethylene, and polyolefine.

50. The kit of claim 43, wherein the heat shrinkage cap is comprised of a material selected from a group consisting of heat shrinkage films of neoprene, teflon, nylon, urethane, silicone, polyvinylchloride, polypropylene, polyethylene, polyethyleneterephthalate, polytetrafluoroethylene, and polyolefine.

51. The kit of claim 43, wherein the heat shrinkage cap is configured to be placed over an open end of an assembly of the first substrate, the second substrate, and the spacer after shrinkage of the bag around a side of the first and second substrates.

52. The kit of claim 47, wherein the spacer comprises a thickness of about 0.2 to about 0.3 mm, a width W3 of about 25 mm of one side, a height H3 of about 64 to about 76 mm of two sides, a segment width W5 of about 2.5 to about 4 mm, and a segment height H5 of about 2.5 to about 5 mm.

53. The kit of claim 47, wherein the spacer comprising a thickness of about 0.2 to about 0.3 mm, a width W3 of about 25 mm of one side, a height H3 of about 64 to about 76 mm of two sides, a segment width W5 of about 2.5 mm, and a segment height H5 of about 2.5 mm, and the first substrate and the second substrate have substantially same rectangular dimensions.

54. The kit of claim 42, wherein the heat shrinkage bag is comprised of a heat shrinkage film of polyvinylchloride (PVC) or polyethyleneterephthalate (PET), and comprises a substantially rectangular structure with one dimension of about 30 mm and another dimension of about 82 to about 100 mm.

55. The kit of claim 42, wherein the spacer is affixed to a surface of the first substrate.

56. The kit of claim 42, wherein the spacer is affixed to a surface of the second substrate.

57. The kit of claim 42, wherein the first substrate and second substrate have substantially rectangular dimensions.
